(19) United States
(12) Patent Application Publication
(10) Pub. No.: US 2004/0220063 A1
(43) Pub. Date: Nov. 4, 2004

(54) Personal cleansing compositions

(76) Inventors: Michael Jahi Chappell, Ann Arbor, MI (US); Mannie Lee Clapp, Mason, OH (US); Magda El-Nokaly, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/368,932

(22) Filed: Feb. 19, 2003

Related U.S. Application Data
(60) Provisional application No. 60/358,225, filed on Feb. 20, 2002.

(51) Int. Cl. A61K 7/50

(52) U.S. Cl. 510/130; 510/470

(57) ABSTRACT

Personal cleansing compositions contain solid anionic particles. More specifically, personal cleansing compositions contain solid anionic particles and a cationic polymer, wherein the anionic particles are complexed with the cationic polymer to form aggregates prior to mixing with the remaining ingredients and wherein the aggregates are present in the finished product. The personal cleansing products can be used to cleanse human skin, hair and nails.

### CROSS REFERENCE TO RELATED APPLICATION

This application claims the benefit of U.S. Provisional Application No. 60/358,225, filed Feb. 20, 2002.

### FIELD OF THE INVENTION

The present invention relates to a personal cleansing composition containing anionic particles. More specifically, it relates to a personal cleansing composition containing anionic particles and a cationic polymer, wherein the anionic particles are complexed with the cationic polymer to form aggregates prior to mixing with the remaining ingredients.

### BACKGROUND OF THE INVENTION

Solid particles are known for use as benefit agents in a variety of formulations and personal care compositions. Solid particles can impart benefits both to the compositions comprising them or surfaces to which the compositions are applied. Solid particles can for example be used as pigments or coloring agents, opacifiers, pearlescent agents, feel modifiers, oil absorbers, skin protectants, matting agents, friction enhancers, slip agents, conditioning agents, exfoliants, odor absorbers, or cleaning enhancers. Additionally, many active ingredients useful as treatment agents for various disorders or socially embarrassing conditions are available and typically used in solid particulate form including antiperspirant agents, anti-dandruff agents, antimicrobials, antibiotics, and sunscreens.

Typically when it is desired to modify the properties of a surface through application of particles, the particles are applied via leave-on preparations, which are rubbed, sprayed, or otherwise applied directly onto the surface to be affected. Typical personal care preparations suitable for delivery of solid particles to hair or skin surfaces include as examples moisturizers, lotions, creams, loose or pressed powders, sticks, tonics, gels, and various sprays such as aerosol or pump sprays. These products are typically applied directly to the surface whereupon particles are deposited and retained by the composition itself or by residual non-volatile elements of the composition after evaporation and drying.

It has also been known to formulate solid particle benefit agents into rinse-off or cleansing compositions such as hair rinses, personal cleansing compositions, liquid and bar soaps, conditioners, or colorants. Frequently the solid particle benefit agent is used to affect the overall appearance, stability or aesthetics of the composition itself. As example, it is well known to add colorant particles, pigments, or pearlescent agents to compositions to improve the acceptability and attractiveness of the product to potential consumers. It is also well known to add particulate benefit agents to affect the in use performance, appearance or aesthetic properties of the composition or to provide a tactile signal to the user. As example, exfoliant particles are frequently used in cleansing compositions to improve abrasion and removal of oils and dirt from washed surfaces and to impart a perceptible “scrubbing” sensation to the user. Typically such solid particle agents are not intended or desired to be deposited onto the substrate and are removed during dilution and rinsing of the composition from the surface to which they are applied.

Given the broad range of benefits, which can be delivered through application and retention of solid particles on surfaces, however, it can be highly desirable to have rinse-off compositions capable of depositing an effective level of solid particles to the surface treated with compositions containing the desired solid particle benefit agent. Compositions intended to deposit solid particle benefit agents to hair or skin surfaces are known; however, the efficiency of deposition has heretofore been unacceptable, requiring either an excess of the solid particle agent in the composition to affect delivery or an unperceivable or unacceptable level of the benefit to be obtained. The efficient deposition and retention of solid particle benefit agents is particularly difficult from compositions intended for cleansing or washing of surfaces, such as personal cleansing products, which contain surfactants and other ingredients which are used to solubilize, suspend and remove particulate and oily substances from the surfaces treated therewith.

It remains, therefore, highly desirable to have a rinse-off composition, preferably a cleansing composition, capable of containing and effectively depositing and retaining solid particle benefit agents on the surface treated therewith. It has now been discovered that select cationic polymers, when used in the cleansing compositions of the present invention, can surprisingly enhance the deposition and retention of solid particle benefit agents on the surfaces treated therewith when they are mixed with the solid particle prior to addition to the remaining composition.

### SUMMARY OF THE INVENTION

The present invention meets the aforementioned needs by providing a personal cleansing composition comprising a cleansing surfactant, from about 0.1% to about 20% by weight of anionic particles, the balance being conventional adjunct personal cleansing ingredients, wherein the anionic particles are complexed with a cationic polymer to form aggregates prior to addition to the remaining composition and wherein the aggregates are present in the finished product. Preferably the anionic particles are selected from the group consisting of silicas, silicates, carbonates, any other silica-containing or non-silica-containing powder suitable for absorbing moisture or oil from the applied surface of the body and mixtures thereof and have a mean particle size of less than about 300 μm.

This invention also provides a personal cleansing composition wherein the anionic particles have a Moisture Absorption Value of at least about 0.5. It is further preferred that the cationic polymer have a charge density of at least about 0.4 meq/gm and less than about 7 meq/gm.

The invention further provides a personal cleansing composition wherein the cationic polymer is selected from the group consisting of polysaccharide polymers having the formula:

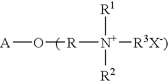

wherein A is an anhydroglucose residual group; R is selected from the group consisting of an alkylene oxyalkylene, polyoxyalkylene, and hydroxyalkylene group, or combinations thereof; R¹, R², and R³ are independently selected from the group consisting of alkyl, aryl, alkylaryl, arylalkyl, alkoxyalkyl, and alkoxyaryl groups, each group containing up to about 18 carbon atoms, and the total number of carbon atoms in R¹, R² and R³ being about 20 or less; and X is an anionic counterion, Polyquaternium 10, Polyquaternium 24, cationic guar gum derivatives, quaternary nitrogen-containing cellulose ethers, copolymers of etherified cellulose, guar and starch and mixtures thereof.

A method of manufacturing a personal cleansing composition comprising the steps of:

a) complexing an anionic particle with a cationic polymer to form aggregates;

b) adding the aggregates to a personal cleansing composition comprising a cleansing surfactant and other conventional personal cleansing adjuncts, the balance being water wherein the anionic particles have a mean particle size of less than about 300 μm is also provided.

Additionally, a method of depositing particles onto human skin, hair or nails comprising the steps of applying the personal cleansing compositions as described herein to the skin and rinsing off is also provided.

All documents cited are hereby incorporated by reference in their entirety.

### DETAILED DESCRIPTION OF THE INVENTION

The present invention achieves the aforementioned benefits by providing a rinse-off personal cleansing composition, which effectively deposits solid particles onto the human skin.

While the specification concludes with claims, which particularly point out and distinctly claim the invention, it is believed the present invention will be better understood from the following description.

The personal cleansing compositions of the present invention include detersive surfactant, anionic particles, a cationic polymer and conventional adjunct personal cleansing ingredients. Each of these components, as well as preferred or optional components, are described in detail hereinafter.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified.

All molecular weights as used herein are weight average molecular weights expressed as grams/mole, unless otherwise specified.

The term “charge density”, as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “fluid” as used herein, means a liquid or a gas.

The term “polymer” as used herein shall include materials whether made by polymerization of one type of monomer or made by two (i.e., copolymers) or more types of monomers.

The term “solid particle” as used herein means a particle that is not a liquid or a gas.

The term “sphere” as used herein, means a spherical body, which is the set of points in a metric space whose distance from a fixed point is approximately constant. Here, the meaning of “approximately” is that the fixed points are within a distance of ±15%.

The term “suitable for application to human skin” as used herein, means that the compositions or components thereof so described are suitable for use in contact with human skin without undue toxicity, incompatibility, instability, allergic response, and the like.

The term “water soluble” as used herein, means that the polymer is soluble in water in the present composition. In general, the polymer should be soluble at 250° C. at a concentration of 0.1% by weight of the water solvent, preferably at 1%, more preferably at 5%, most preferably at 15%.

Anionic Particles

The personal cleansing compositions of the present invention comprise solid anionic particles suspended or dispersed throughout the compositions. Particles are defined as anionic if they have a negative zeta potential as defined below. Zeta potentials are determined by using a Brookhaven Zeta Plus Zeta potential analyzer. A dilute suspension of particles (i.e. 0.1 g particles in 25 g deionized (DI) water) is first prepared, then 1 to 2 drops of this suspension is diluted in 10 mM KCI. The pH of the system is not adjusted. Zeta potential analysis is performed on the sample diluted in KCI. For the purposes of this invention, particles are defined as anionic if the mean of 10 runs results in a negative zeta potential.

The solid anionic particles for use in the compositions of the present invention include moisture-absorbent materials such as silicas (or silicon dioxides), silicates, carbonates, and combinations thereof. The silicates are most typically those formed by reaction of a carbonate or silcate with an alkali metal, alkaline earth metal, or transition metal, specific non-limiting examples of which include calcium silicate, amorphous silicas (e.g., precipitated, fumed, gel and colloidal), calcium carbonate (e.g., chalk), magnesium carbonate, zinc carbonate, and combinations thereof. Non-limiting examples of some suitable silicates and carbonates for use herein are described in Van Nostrand Reinhold's Encyclopedia of Chemistry, 4^{th} edition, pages 155, 169, 556, and 849 (1984), which descriptions are incorporated herein by reference. Absorbent powders are also described in U.S. Pat. No. 6,004,584 (Peterson et al.), which description is incorporated herein by reference.

The average particle size of the solid anionic particles for use in the compositions is preferably less than about 300 μm. Preferably the solid anionic particles will have a particle size of from about 0.1μm to about 80 μm, more preferably from about 0.1 μm to about 70 μm and even more preferably from about 1 μm to about 60 μm. It has been found that the visible residue of such solid materials is greatly reduced when the average particle size of the solid materials is reduced. Typical processes used to reduce the particle size sufficiently include wet milling and controlled flow cavitation. In a wet milling process, a slurry is prepared with the particle to be reduced and water or other suitable fluid. The slurry is placed in a stirred media mill chamber with potential dispersants. The dispersants could be ceramic, stainless steel, polymeric coated materials or other and may range in size from 50 microns to 3 millimeters. The tip speed of the rotor arms may range between 5 to 20 meters per second and total particle residence time may vary from 30 to 300 seconds. Netzsch is a manufacturer of these types of stirred media mills.

The solid anionic particles remain solid within the composition and preferably provide fluid absorption properties when applied topically to the skin. Among the solid anionic particles for use herein, highly preferred are those that have a Moisture Absorption Value of at least about 0.5, preferably at least about 1.0, more preferably at least about 2.0, even more preferably at least about 2.5 grams of moisture absorption per gram of solid anionic particle as measured in accordance with the Moisture Absorption Test as described herein. These Moisture Absorption Values have been found to correlate with the ability of the topical compositions of the present invention containing such materials to provide moisture, sweat and/or sebum absorption from the applied surface over extended periods of time after topical application.

The concentration of solid anionic particles in the composition preferably ranges from about 0.1% to about 20%, more preferably from about 0.5% to about 10%, even more preferably from about 2% to about 8%, by weight of the composition.

It has been found, therefore, that the solid anionic particles as described herein can be formulated into the compositions of the present invention to provide long lasting or enduring moisture, sebum and/or sweat absorption from the skin or other applied area after application.

Moisture Absorption Test

The solid anionic particles for use in the compositions of the present invention preferably have a minimum Moisture Absorption Value of at least about 0.5 gram/gram, wherein the Moisture Absorption Value is determined in accordance with the following Moisture Absorption Test.

A powder chamber (Kruss Fiber Cell) and two filter papers (Kruss filter paper, part # FL12PLP) are preweighed on a balance and balance tared. One of the filter papers is then placed at the bottom of the powder chamber. The sample of absorbent powder to be tested is then packed into the chamber using a spatula. The mass of powder loaded into the chamber will vary depending upon the density of the powder, but will most typically range from about 0.50 grams to about 3.5 grams of powder packed into the powder chamber. A second filter paper is then placed on top of the packed powder and the screw cap for the chamber is placed on the chamber. The chamber knob is then rotated by hand until the powder is firmly packed and the knob can no longer be rotated manually. The powder chamber is then placed within a Kruss Tensiometer. The Tensiometer glass dish is then filled with distilled water and positioned on the stage device. The Tensiometer is then turned on and the stage is raised to just below the powder chamber so that the powder does not yet contact the distilled water in the dish. The Tensiometer is then balanced and allowed to tare. The stage is raised until the powder chamber is immersed 9 mm in the distilled water. The mass of the powder sample is measured every fifteen seconds until the mass has reached equilibrium and no longer fluctuates drastically.

The Water Absorption Value is then determined for any given sample by calculating the difference between the mass of the powder chamber at 15 seconds and the mass of the powder chamber at the equilibrium point, divided by the mass of powder initially loaded into the chamber.

Cationic Polymer

The compositions of the present invention include a cationic deposition polymer of sufficiently high cationic charge density to effectively enhance deposition of the solid particle component described herein. Suitable cationic polymers will have cationic charge densities of at least about 0.4 meq/gm, preferably at least about 0.7 meq/gm, more preferably at least about 0.9 meq/gm, but also preferably less than about 7 meq/gm, more preferably less than about 5 meq/gm, at the pH of intended use of the personal cleansing composition, which pH will generally range from about pH 3 to about pH 9, preferably between about pH 4 and about pH 8. The average molecular weight of such suitable cationic polymers will generally be between about 10,000 and 10 million, preferably between about 50,000 and about 5 million, more preferably between about 100,000 and about 3 million. The “cationic charge density” of a polymer, as that term is used herein, refers to the ratio of the number of positive charges on a monomeric unit of which the polymer is comprised to the molecular weight of said monomeric unit. The cationic charge density multiplied by the polymer molecular weight determines the number of positively charged sites on a given polymer chain.

The concentration of the cationic polymer in the personal cleansing composition ranges from about 0.05% to about 3%, preferably from about 0.075% to about 2.0%, more preferably from about 0.1% to about 1.0%, by weight of the personal cleansing composition. The weight ratio of cationic polymer to solid anionic particle in the personal cleansing compositions is from about 2:1 to about 1:100, preferably from about 1:1 to about 1:50, more preferably from about 1:1 to about 1:30.

The cationic polymer for use in the personal cleansing composition of the present invention contains cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the personal cleansing composition. Any anionic counterions can be used in association with the cationic polymers so long as the polymers remain soluble in water, in the personal cleansing composition, or in a coacervate phase of the personal cleansing composition, and so long as the counterions are physically and chemically compatible with the essential components of the personal cleansing composition or do not otherwise unduly impair product performance, stability or aesthetics. Non limiting examples of such counterions include halides (e.g., chlorine, fluorine, bromine, iodine), sulfate and methylsulfate.

The cationic nitrogen-containing moiety of the cationic polymer is generally present as a substituent on all, or more typically on some, of the monomer units thereof. Thus, the cationic polymer for use in the personal cleansing composition includes homopolymers, copolymers, terpolymers, and so forth, of quaternary ammonium or cationic amine-substituted monomer units, optionally in combination with non-cationic monomers referred to herein as spacer monomers. Non limiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which description is incorporated herein by reference.

Cationic polymers for use in the personal cleaning compositions described herein will preferably have a settling time of less than the settling time of the same compositions without the polymer added, as determined by the settling test described below.

Settling test

4 grams of neat product is placed into a 50 ml conical bottom centrifuge tube (Corning part # 430304 or similar) and diluted with 36 grams of distilled water. The tube is then capped and shaken vigorously until all of the product has been dispersed. This is considered the initial time. The tube is then let stand vertically. For products containing powder, the system will be opaque due to the suspension of the powder. To screen polymer systems, batches with and without polymer are made and compared in this test. Preferred polymers result in a settling of the particles on a rapid time scale, generally on the order of less than 5 minutes, however, any settling time less than that of the same composition without polymer indicates a suitable polymer. Settling can be observed by the ability to read markings on the back of the centrifuge tube through the top half of the sample, which will initially not be visible due to the cloudiness of the product. The time taken for this to occur is considered the settling time.

Suitable cationic polymers for use in the personal cleansing composition include polysaccharide polymers, such as cationic cellulose derivatives and cationic starch derivatives. Suitable cationic polysaccharide polymers include those, which conform to the formula:

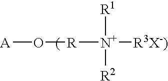

wherein A is an anhydroglucose residual group, such as a starch or cellulose anhydroglucose residual; R is an alkylene oxyalkylene, polyoxyalkylene, or hydroxyalkylene group, or combination thereof; R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less; and X is an anionic counterion, as previously described. The degree of cationic substitution in these polysaccharide polymers, is typically from about 0.0 1-1 cationic groups per anhydroglucose unit.

Preferred cationic cellulose polymers salts of hydroxyethyl cellulose reacted with trimethyl ammonium substituted epoxide, referred to in the industry (CTFA) as Polyquaternium 10 and available from Amerchol Corp. (Edison, N.J., USA) as Polymer JR30M with charge density of 1.25 meq/g and molecular weight of ˜900,000, Polymer JR400 with charge density of 1.25 meq/g and molecular weight of ˜400,000, and Polymer KG30M with a charge density of 1.9 and a molecular weight of ˜1.25 million. Other suitable types of cationic cellulose includes the polymeric quaternary ammonium salts of hydroxyethyl cellulose reacted with lauryl dimethyl ammonium-substituted epoxide referred to in the industry (CTFA) as Polyquaternium 24.

Other suitable cationic polymers include cationic guar gum derivatives, such as guar hydroxypropyltrimonium chloride, a specific examples of which includes Jaguar C17 with a charge density of 0.9 and molecular weight of ˜2.2 million commercially available from Rhone-Poulenc Incorporated . Other suitable cationic polymers include quaternary nitrogen-containing cellulose ethers, some examples of which are described in U.S. Pat. No. 3,962,418, which description is incorporated herein by reference herein. Other suitable cationic polymers include copolymers of etherified cellulose, guar and starch, some examples of which are described in U.S. Pat. No. 3,958,581, which description is incorporated herein by reference.

Cleansing Surfactant

The personal cleansing compositions of the present invention comprise a surfactant suitable for application to the hair or skin. Suitable surfactants for use herein include any known or otherwise effective cleansing surfactant suitable for application to the hair or skin, and which is otherwise compatible with the other essential ingredients in the compositions. Suitable cleansing surfactants include anionic, nonionic, cationic, zwitterionic or amphoteric surfactants, or combinations thereof.

The personal cleansing compositions of the present invention preferably comprise from about 0.1% to about 50%, more preferably from about 4% to about 30%, even more preferably from about 5% to about 25%, by weight of the composition of cleansing surfactant.

Anionic surfactants suitable for use in the personal cleansing compositions include alkyl and alkyl ether sulfates. These materials have the respective formulae ROSO₃M and RO(C₂H₄O)ₓSO₃M, wherein R is alkyl or alkenyl of from about 8 to about 24 carbon atoms, x is 1 to 10, and M is a water-soluble cation such as ammonium, sodium, potassium or triethanolamine. The alkyl ether sulfates are typically made as condensation products of ethylene oxide and monohydric alcohol's having from about 8 to about 24 carbon atoms. Preferably, R has from about 10 to about 18 carbon atoms in both the alkyl and alkyl ether sulfates. The alcohol's can be derived from fats, e.g., coconut oil or tallow, or can be synthetic. Lauryl alcohol and straight chain alcohol's derived from coconut oil are preferred herein. Such alcohol's are reacted with about 1 to about 10, preferably from about 3 to about 5, and especially about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the personal cleansing compositions include are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [ R¹-SO₃-M ], wherein R¹ is chosen from the group consisting of a straight or branched chain, saturated aliphatic hydrocarbon radical having from about 8 to about 24, preferably about 10 to about 18, carbon atoms; and M is a cation. Preferred examples include the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀₋₁₈ n-paraffins.

Additional examples of suitable anionic surfactants are the reaction products of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil. Other suitable anionic surfactants of this variety are described in U.S. Pat. No. 2,486,921; U.S. Pat. No. 2,486,922; and U.S. Pat. No. 2,396,278; which descriptions are incorporated herein by reference.

Still other suitable anionic surfactants are the succinamates, examples of which include disodium N-octadecylsulfosuccinamate; diammoniumlauryl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having from about 12 to about 24 carbon atoms. The term “olefin sulfonates” is used herein to mean compounds which can be produced by the sulfonation of a-olefins by means of uncomplexed sulfur trioxide, followed by neutralization of the acid reaction mixture in conditions such that any sulfones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form.

The a-olefins from which the olefin sulfonates are derived are mono-olefins having about 12 to about 24 carbon atoms, preferably about 14 to about 16 carbon atoms. Preferably, they are straight chain olefins.

In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process.

Another class of anionic surfactants suitable for use in the personal cleansing compositions of the present invention is the b-alkyloxy alkane sulfonates, which conform to the formula:

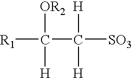

wherein R¹ is a straight chain alkyl group having from about 6 to about 20 carbon atoms, R₂ is a lower alkyl group having from about 1 to about 3 carbon atoms, preferably 1 carbon atom, and M is a water-soluble cation.

Other suitable surfactants for use in the personal cleansing compositions herein are described in McCutcheon's, Emulsifiers and Detergents, 1989 Annual, published by M. C. Publishing Co., and in U.S. Pat. No. 3,929,678, which descriptions are incorporated herein by reference.

Preferred anionic surfactants for use in the personal cleansing compositions herein include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Amphoteric surfactants suitable for use in the personal cleansing compositions herein include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Non-limiting examples of such surfactants include sodium 3-dodecyl-aminopropionate, sodium 3-dodecylaminopropane sulfonate, sodium lauryl sarcosinate, N-alkyltaurines such as those prepared by reacting dodecylamine with sodium isethionate according to the teaching of U.S. Pat. No. 2,658,072, N-higher alkyl aspartic acids such as those prepared in accordance with the teaching of U.S. Pat. No. 2,438,091, and the products described in U.S. Pat. No. 2,528,378, which teachings and descriptions are incorporated herein by reference.

Other suitable amphoteric surfactants include the alkali, alkaline earth, ammonium and trialkanolammonium salts of cocoamphoacetate, cocoamphodiacetate, cocoamphopropionate, cocoamphodipropionate, amphoacetates such as lauroamphoacetate or cocoamphoacetate and mixtures thereof.

Also suitable are soaps—mono and divalent salts of fatty acids.

Cationic surfactants can also be used in the personal cleansing compositions herein, but are generally less preferred, and preferably represent less than about 5% by weight of the compositions.

Suitable nonionic surfactants for use in the personal cleansing compositions herein include condensation products of alkylene oxide groups with an organic hydrophobic compound, which may be aliphatic or alkyl aromatic in nature. Preferred classes of nonionic surfactants include: 1) polyethylene oxide condensates of alkyl phenols, e.g., the condensation products of alkyl phenols having an alkyl group containing from about 6 to about 20 carbon atoms in either a straight chain or branched chain configuration, with ethylene oxide, the ethylene oxide being present in amounts equal to from about 10 to about 60 moles of ethylene oxide per mole of alkyl phenol; 2) nonionic surfactants derived from the condensation of ethylene oxide with the product resulting from the reaction of propylene oxide and ethylene diamine products; 3) condensation products of aliphatic alcohol's having from about 8 to about 18 carbon atoms, in either straight chain or branched chain configuration, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms; 4) long chain tertiary amine oxides corresponding to the following general formula:

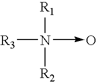

wherein R₁ contains an alkyl, alkenyl or monohydroxy alkyl radical of from about 8 to about 18 carbon atoms, from 0 to about 10 ethylene oxide moieties, and from 0 to about I glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals; 5) long chain tertiary phosphine oxides corresponding to the following general formula:

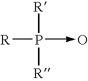

wherein R contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms; 6) long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety; 7) alkyl polysaccharide (APS) surfactants such as the alkyl polyglycosides, as described in U.S. Pat. No. 4,565,647, which have a hydrophobic group with about 6 to about 30 carbon atoms and polysaccharide (e.g., polyglycoside) as the hydrophilic group, and optionally have a polyalkylene-oxide group joining the hydrophobic and hydrophilic moieties, wherein the alkyl group (i.e., the hydrophobic moiety) can be saturated or unsaturated, branched or unbranched, and unsubstituted or substituted (e.g., with hydroxy or cyclic rings); and 8) polyethylene glycol (PEG) glyceryl fatty esters, such as those of the formula R(O)OCH₂CH(OH)CH₂(OCH₂CH₂)ₙOH wherein n is from about 5 to about 200, preferably from about 20 to about 100, and R is an aliphatic hydrocarbyl having from about 8 to about 20 carbon atoms.

Zwitterionic surfactants suitable for use in the personal cleansing compositions herein include those that are broadly described as derivatives of aliphatic quaternary ammonium, phos-phonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phos-phonate. These zwitterionic surfactants include those represented by the formula:

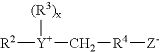

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about I to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use in the personal cleansing compositions herein include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Conventional Adjunct Ingredients

The personal cleansing compositions of the present invention may further comprise other adjunct ingredients that may modify the physical, chemical, cosmetic or aesthetic characteristics of the compositions or serve as additional “active” components when deposited on the skin. The compositions may also further comprise adjunct inert ingredients. Many such adjunct ingredients are known for use in personal care compositions, and may also be used in the topical compositions herein, provided that such adjunct materials are compatible with the essential materials described herein, or do not otherwise unduly impair product performance.

Such adjunct ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. Non limiting examples of such adjunct ingredients include preservatives (e.g., propyl paraben), deodorants, antimicrobials, fragrances, deodorant perfumes, coloring agents or dyes, thickeners, sensates, sunscreens, surfactants or emulsifiers, gellants or other suspending agents, pH modifiers, co-solvents or other additional solvents, emollients, pharmaceutical actives, vitamins, and combinations thereof.

Other adjunct materials include perfumes or fragrances, including deodorant and pro-perfumes, concentrations of which optionally typically range from about 0.1% to about 5%, more typically from about 0.5% to about 4%, by weight of the composition.

The personal cleansing compositions of the present invention may further comprise a stabilizing agent at concentrations effective for stabilizing the particle,. or other water-insoluble material, in dispersed form in the personal cleansing compositions or for modifying the viscosity of the composition. Such concentrations range from about 0.1% to about 10%, preferably from about 0.3% to about 5.0%, by weight of the personal cleansing compositions.

Stabilizing agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Polyalkylene glycols having a molecular weight of more than about 1000 are useful herein. Useful are those having the following general formula:

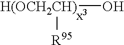

wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, x3 has an average value of from about 1500 to about 25,000, preferably from about 2500 to about 20,000, and more preferably from about 3500 to about 15,000. Other useful polymers include the polypropylene glycols and mixed polyethylene-polypropylene glycols, or polyoxyethylene-polyoxypropylene copolymer polymers,. Polyethylene glycol polymers useful herein are PEG-2M wherein R⁹⁵ equals H and x3 has an average value of about 2,000 (PEG-2M is also known as Polyox WSR® N-10, which is available from Union Carbide and as PEG-2,000); PEG-5M wherein R⁹⁵ equals H and x3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and x3 has an average value of about 7,000 (PEG-7M is also known as Polyox WSR® N-750 available from Union Carbide); PEG-9M wherein R⁹⁵ equals H and x3 has an average value of about 9,000 (PEG 9-M is also known as Polyox WSR® N-3333 available from Union Carbide); and PEG-14 M wherein R⁹⁵ equals H and x3 has an average value of about 14,000 (PEG-14M is also known as Polyox WSR® N-3000 available from Union Carbide).

Commercially available viscosity modifiers highly useful herein include Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Hercules, ethylene oxide and/or propylene oxide based polymers with tradenames CARBOWAX PEGs, POLYOX WASRs, and UCON FLUIDS, all supplied by Amerchol.

Other adjunct stabilizing agents include crystalline stabilizing agents which can be categorized as acyl derivatives, long chain amine oxides, and mixtures thereof. These stabilizing agents are described in U.S. Pat. No. 4,741,855, which description is incorporated herein by reference. These preferred stabilizing agents include ethylene glycol esters of fatty acids preferably having from about 16 to about 22 carbon atoms. More preferred are the ethylene glycol stearates, both mono and distearate, but particularly the distearate containing less than about 7% of the mono stearate. Other suitable stabilizing agents include alkanol amides of fatty acids, preferably having from about 16 to about 22 carbon atoms, more preferably about 16 to 18 carbon atoms, preferred examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids in addition to the preferred materials listed above may be used as stabilizing agents.

Other long chain acyl derivatives suitable for use as stabilizing agents include N,N-dihydrocarbyl amido benzoic acid and soluble salts thereof (e.g., Na, K), particularly N,N-di(hydrogenated) C.sub.16, C.sub.18 and tallow amido benzoic acid species of this family, which are commercially available from Stepan Company (Northfield, Ill., USA).

Examples of suitable long chain amine oxides for use as stabilizing agents include alkyl (C.sub.16 -C.sub.22) dimethyl amine oxides, e.g., stearyl dimethyl amine oxide. Other suitable stabilizing agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable stabilizing agents include di(hydrogenated tallow)phthalic acid amide, and crosslinked maleic anhydride-methyl vinyl ether copolymer.

Other suitable stabilizing agents include crystalline, hydroxyl-containing stabilizers. These stabilizers can be hydroxyl-containing fatty acids, fatty esters or fatty soap water-insoluble wax-like substances or the like. If present, crystalline, hydroxyl-containing stabilizers may comprise from about 0.5% to about 10%, preferably from about 0.75% to about 8%, more preferably from about 1.25% to about 5% by weight of the compositions herein. The said stabilizer is insoluble in water under ambient to near ambient conditions.

Suitable crystalline, hydroxyl-containing stabilizers include:

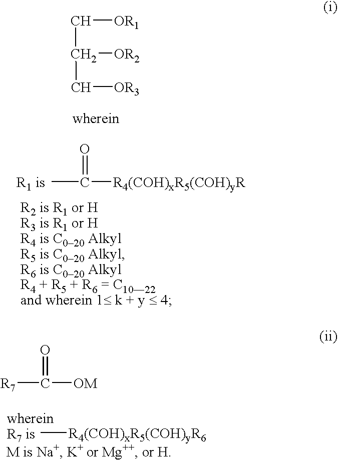

Some preferred hydroxyl-containing stabilizers include 12-hydroxystearic acid, 9,10-dihydroxystearic acid, tri-9,10-dihydroxystearin and tri-12-hydroxystearin (hydrogenated castor oil is mostly tri-12-hydroxystearin). Tri-12-hydroxystearin is most preferred for use in the compositions herein.

The adjunct ingredients as described herein shall specifically exclude, however, any essential ingredient or material as otherwise described or defined herein. However, it should be understood that compositions according to the present invention may contain additional cationic polymers as adjunct ingredients separate from the polymer that is premixed with the anionic particles.

Optional Particulates

The compositions of the present invention may further comprise particulates other than and in addition to the solid anionic particles described herein. These optional particulates can be dispersed throughout the composition to allow for deposition onto the hair, nails or skin after topical application of the composition. The optional particulate can be any skin active agent or other material known for or otherwise useful in application to and deposition onto the hair, nails or skin. Such particulates include materials such as emollients, perfumes, vitamins, sunscreens, pigments or colorants, pharmaceuticals or other skin active agents, or any other material that provides a cosmetic, skin active, or other consumer desirable benefit when applied to and deposited on the hair, nails, or skin.

Method of Manufacture

The present invention also relates to a method of manufacturing a personal cleansing composition. To achieve improved deposition of the anionic particles onto the skin, the anionic particles and the cationic polymer must be mixed together and allowed to form aggregates. It is necessary to first hydrate the cationic polymer with distilled water, then add the anionic particles to the cationic polymer and agitate until the dispersion is smooth. In a separate vessel mix the surfactants and adjunct ingredients and then add to the particle/polymer dispersion. pH and viscosity can at this time be adjusted by conventional means. The aggregates of the anionic particles and the cationic polymer will be visible, in the finished product, under a microscope.

Method of Use

The personal cleansing compositions of the present invention are used in a conventional manner for cleansing hair or skin and providing enhanced deposition of solid particles and other benefits of the present invention. An effective amount of the composition for cleansing the hair or skin is applied to the hair or skin that has preferably been wetted with water, and then rinsed off. Such effective amounts generally range from about 1 g to about 50 g, preferably from about 1 g to about 20 g.

This method for cleansing the hair and skin comprises the steps of:

a) wetting the hair and/or skin with water, b) applying an effective amount of the personal cleansing composition to the hair and/or skin, and c) rinsing the composition from the hair and/or skin using water. These steps can be repeated as many times as desired to achieve the desired cleansing and particle deposition benefits.

The following examples further describe and demonstrate the preferred embodiments within the scope of the present invention. The examples are given solely for the purpose of illustration, and are not to be construed as limitations of the present invention since many variations thereof are possible without departing from its scope.

### EXAMPLES

TABLE 1
Examples 1-6

| | | | Comparative | | Comparative | | Comparative |
|---|---|---|---|---|---|---|---|
| | | | Example | | Example | | Example |
| | Component | 1 | 2 | 3 | 4 | 5 | 6 |
| 1 | Sodium lauryl | 6 | 6 | 6 | 6 | 5.80 | 6 |
| | ether 3EO | | | | | | |
| | sulfate | | | | | | |
| 2 | Cocoamidopropyl | 5 | 5 | 5 | 5 | 5.15 | 5 |
| | Betaine | | | | | | |
| 3 | Sodium Lauroyl | 0.5 | 0.5 | 0.5 | 0.5 | 0.5 | 0.5 |
| | Sarcosinate | | | | | | |
| 4 | Silica Syloid | 5 | 5 | 5 | 5 | 5 | 5 |
| | 244 | | | | | | |
| 5 | Fragrance | 0.5 | 0.5 | 0.5 | 0.5 | 0.7 | 0.5 |
| 6 | Glydant | 0.21 | 0.21 | 0.21 | 0.21 | 0.3 | 0.21 |
| 7 | Ucare KG-30M | 0.25 | | | | 0.25 | |
| 8 | Polycare 133 | | 0.25 | | | | |
| 9 | Jaguar C-17 | | | 0.25 | | | |
| 10 | Sensomer CI-50 | | | | 0.25 | | |
| 11 | Water | QS | QS | QS | QS | QS | QS |
| 12 | Sodium Sulfate | 1 | 1 | 1 | 1 | 1 | 1 |
| 13 | Citric Acid | Adjust | Adjust ph | Adjust | Adjust ph | Adjust | Adjust ph |
| | | ph | | ph | | ph | |
| 14 | EDTA | | | | | 0.15 | |
| 15 | Trihydroxystearin | | | | | 1.5 | |
| 16 | Lauric Acid | | | | | 0.5 | |
| | Aggregates | Y | | Y | | Y | |
| | Present in | | N | | N | | N |
| | Product? | | | | | | |
| | Settling time | <5 | >60 | <5 | <5 | <5 | >60 |
| | (minutes) | | | | | | |

Method of manufacture:
1. Hydrate the polymer (7, 8, 9, or 10) in the distilled water (11)
2. Add silica (4) to polymer and agitate until dispersion is smooth
3. Premix surfactants (1, 2, and 3) in a separate vessel and raise the temperature to about 88° C. (190° F.) and add to polymer/water/silica mixture
4. Add adjunct ingredients when the mixture has cooled to below 60° C. (140° F.) adjust pH if necessary with citric acid, continue cooling to about 38° C. (100° F.)
5. Adjust viscosity, if necessary, with sodium sulfate.
6.

TABLE 2
Examples 7-10

| Component | 7 | 8 | 9 | 10 |
|---|---|---|---|---|
| Sodium lauryl ether 3EO sulfate | 6 | 6 | 6 | 6 |
| Cocoamidopropyl Betaine | 5 | 5 | 5 | 5 |
| Sodium Lauroyl Sarcosinate | 0.5 | 0.5 | 0.5 | 0.5 |
| Silica Sipernat 22LS (Degussa) | 5 | 5 | | |
| Calcium Silicate Hubersorb 600 (Huber) | | | 3 | 3 |
| Fragrance | 0.5 | 0.5 | 0.5 | 0.5 |
| Glydant | 0.22 | 0.22 | 0.22 | 0.22 |
| Ucare JR-30M | 0.25 | | 0.25 | |
| Aqualon Nhance 3196 | | 0.25 | | 0.25 |
| Water | QS | QS | QS | QS |
| Sodium Sulfate | 1 | 1 | 1 | 1 |
| Aggregates Present in Product? | Y | Y | Y | Y |
| Rapid Settling of polymer upon Dilution | Y | Y | Y | Y |

What is claimed is:

1. A personal cleansing composition comprising:
a) a cleansing surfactant;
b) from about 0. 1% to about 20% by weight of anionic particles;
c) the balance conventional adjunct personal cleansing ingredients; wherein the anionic particles are complexed with a cationic polymer to form aggregates prior to addition to the remaining composition and wherein the aggregates are present in the finished product.

2. A personal cleansing composition according to claim 1 wherein the anionic particles are selected from the group consisting of silicas, silicates, carbonates, any other silica-containing or non-silica-containing powder suitable for absorbing moisture or oil and mixtures thereof.

3. A personal cleansing composition according to claim 1 wherein the anionic particles have a mean particle size of less than about 300 μm.

4. A personal cleansing composition according to claim 1 wherein the anionic particles have a Moisture Absorption Value of at least about 0.5.

5. A personal cleansing composition according to claim 1 wherein the cationic polymer has a charge density of at least about 0.4 meq/gm and less than about 7 meq/gm.

6. A personal cleansing composition according to claim 1 wherein the composition with the cationic polymer has a settling time of less than the same composition without the cationic polymer.

7. A personal cleansing composition according to the last claim wherein the cationic polymer is selected from the group consisting of polysaccharide polymers having the formula: 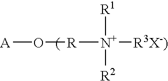
wherein A is an anhydroglucose residual group; R is selected from the group consisting of an alkylene oxyalkylene, polyoxyalkylene, and hydroxyalkylene group, or combinations thereof; R¹, R², and R³ are independently selected from the group consisting of alkyl, aryl, alkylaryl, arylalkyl, alkoxyalkyl, and alkoxyaryl groups, each group containing up to about 18 carbon atoms, and the total number of carbon atoms in R¹, R² and R³ being about 20 or less; and X is an anionic counterion, Polyquaternium 10, Polyquaternium 24, cationic guar gum derivatives, quaternary nitrogen-containing cellulose ethers, copolymers of etherified cellulose, guar and starch and mixtures thereof.

8. A personal cleansing composition comprising:
a) a cleansing surfactant;
b) from about 0.1% to about 20% by weight of anionic particles having a Moisture Absorption Value of at least about 0.5;
c) the balance conventional adjunct personal cleansing ingredients; wherein the anionic particles are complexed with a cationic polymer to form aggregates prior to addition to the remaining composition and wherein the composition with the cationic polymer has a settling time of less than the same composition without the cationic polymer.

9. A method of manufacturing a personal cleansing composition comprising the steps of:
a) complexing an anionic particle with a cationic polymer to form aggregates;
b) adding the aggregates to a personal cleansing composition comprising a cleansing surfactant and other conventional personal cleansing adjuncts, the balance being water wherein the anionic particles have a mean particle size of less than about 300 μm.

10. A method of manufacturing a personal cleansing composition according to claim 9 wherein the anionic particles are selected from the group consisting of silicas, silicates, carbonates, organic copolymers, kaolin, mica, talc, starch, modified starch, microcrystalline cellulose, fluid-absorbent polyethylenes or other functionally similar fluid-absorbent polymer, any other silica-containing or non-silica-containing powder suitable for absorbing moisture or oil and mixtures thereof.

11. A method of manufacturing a personal cleansing composition according to claim 9 wherein the anionic particles have a mean particle size of less than about 300 μm.

12. A method of manufacturing a personal cleansing composition according to claim 9 wherein the anionic particles have a Moisture Absorption Value of at least about 0.5.

13. A method of manufacturing a personal cleansing composition according to claim 9 wherein the cationic polymer has a charge density of at least about 0.4 meq/gm and less than about 7 meq/gm.

14. A method of manufacturing a personal cleansing composition according to claim 9 wherein the composition with the cationic polymer has a settling time of less than the same composition without the cationic polymer.

15. A method of manufacturing a personal cleansing composition according to claim 9 wherein the cationic polymer is selected from the group consisting of polysaccharide polymers having the formula: 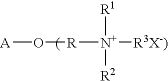
wherein A is an anhydroglucose residual group; R is selected from the group consisting of an alkylene oxyalkylene, polyoxyalkylene, and hydroxyalkylene group, or combinations thereof, R¹, R², and R³ are independently selected from the group consisting of alkyl, aryl, alkylaryl, arylalkyl, alkoxyalkyl, and alkoxyaryl groups, each group containing up to about 18 carbon atoms, and the total number of carbon atoms in R¹, R² and R³ being about 20 or less; and X is an anionic counterion, Polyquaternium 10, Polyquaternium 24, cationic guar gum derivatives, quaternary nitrogen-containing cellulose ethers, copolymers of etherified cellulose, guar and starch and mixtures thereof.

16. A method of depositing particles onto human skin comprising the steps of applying the personal cleansing compositions according to claim 1 to the skin and subsequently rinsing the compositions off.
